(19) United States
(12) Patent Application Publication
(10) Pub. No.: US 2024/0197473 A1
(43) Pub. Date: Jun. 20, 2024

(54) SYSTEMS AND METHODS TO OCCLUDE A VALVULAR COMMISSURE OR CLEFT

(71) Applicant: EDWARDS LIFESCIENCES CORPORATION, Irvine, CA (US)

(72) Inventor: Mark Chau, Laguna Hills, CA (US)

(21) Appl. No.: 18/590,740

(22) Filed: Feb. 28, 2024

Related U.S. Application Data
(63) Continuation of application No. PCT/US2022/042201, filed on Aug. 31, 2022.
(60) Provisional application No. 63/248,210, filed on Sep. 24, 2021.

(51) Int. Cl. A61F 2/24

(52) U.S. Cl. CPC A61F 2/2457; A61F 2210/0004; A61F 2220/0008; A61F 2230/0067; A61F 2230/0091

(57) ABSTRACT

Systems, and methods for treating valvular regurgitation, prolapse, and other valve problems are described. Some embodiments include occluding and/or obstructing devices that traverse an annulus, some implementations anchor an occluding device via a coil. Some implementations provide systems and methods to bring native leaflets in closer proximity to each other to prevent valvular problems. Some embodiments provide outflow-side solutions that encircle chordae tendineae, while some implementations provide inflow-side solutions via joined tissue anchors that can be cinched together.

### CROSS-REFERENCE TO RELATED APPLICATIONS

This application is a continuation of International Patent Application No. PCT/US2022/042201, filed Aug. 31, 2022, which claims the benefit of U.S. Patent Application No. 63/248,210, filed Sep. 24, 2021, the entire disclosures all of which are incorporated by reference for all purposes.

### BACKGROUND

Safe and effective devices, systems, techniques, and methods, for the correction of valvular regurgitation, including mitral regurgitation are desirable. Attempting to address valvular regurgitation only at a central location in the valve, may not properly address regurgitation that occurs at the edges or commissural positions of a valve.

### SUMMARY

This summary is meant to provide examples and is not intended to be limiting of the scope of the invention in any way. For example, any feature included in an example of this summary is not required by the claims, unless the claims explicitly recite the feature. Also, the features described can be combined in a variety of ways. Various features and steps as described elsewhere in this disclosure can be included in the examples summarized here.

In some implementations, an occluding device for preventing regurgitation or prolapse at a native heart valve includes a plug. In some implementations, the plug includes a generally conical shape. In some implementations, the generally conical shape defines a proximal side defining a base of the generally conical shape and a distal side defining a narrow end of the generally conical shape and opposite the base, and a central axis running between the distal and proximal sides, and a coil possessing at least one turn extending along the central axis; wherein the coil is sized to encircle native chordae tendineae of a heart valve and provide a retention force against the central plug.

In some implementations, the coil is joined to the central plug at the distal side.

In some implementations, the coil is joined to the central plug at an outer diameter of the proximal side.

In some implementations, the central plug further comprises a flange extending from the distal tip.

In some implementations, the central plug traverses a native annulus, such that the proximal side resides on the inflow side of a native annulus, and the distal side resides on the outflow side of the native annulus.

In some implementations, the coil possesses at least two turns.

In some implementations, the coil is constructed of a memory material.

In some implementations, the central plug is constructed of a memory material.

In some implementations, the memory material is nitinol.

In some implementations, the central plug comprises a covering.

In some implementations, the covering comprises a biocompatible or atraumatic material.

In some implementations, the covering is constructed of ePTFE, bovine pericardium, porcine pericardium, equine pericardium, woven PTFE, knitted PTFE, braided PTFE, polyurethane, electrospun ePTFE, dipped thermoplastic, sprayed thermoplastic, other organic tissues, other non-organic tissues, and combinations thereof.

In some implementations, the covering comprises a material or compound to encourage tissue ingrowth.

In some implementations again, the coil and the central plug possess a braided weave.

In some implementations, the occluding device further includes a lubricous outer surface.

In some implementations, the lubricous outer surface is disposed on the coil.

In some implementations, the lubricous outer surface is disposed on the central plug.

In some implementations, the lubricous outer surface is constructed of a bioabsorbable material.

In some implementations, an occluding device for preventing regurgitation at a native heart valve includes a central plug having a generally conical shape, wherein the generally conical shape defines a proximal side being the base of the conical shape and a distal side defining the narrow end opposite the base and a central axis running between the distal and proximal sides, and a plurality of tissue anchors extending radially from the distal side and sized to capture valvular leaflets against the central plug.

In some implementations, the plurality of tissue anchors possess a contoured shape.

In some implementations, the contoured shape is comprised of a covering disposed upon the plurality of tissue anchors.

In some implementations, the central plug is formed of a memory material.

In some implementations, the central plug further comprises a biocompatible or atraumatic material.

In some implementations, the biocompatible or atraumatic material or covering is selected from: ePTFE, bovine pericardium, porcine pericardium, equine pericardium, woven PTFE, knitted PTFE, braided PTFE, polyurethane, electrospun ePTFE, dipped thermoplastic, sprayed thermoplastic, other organic tissues, other non-organic tissues, and combinations thereof.

In some implementations, the plurality of tissue anchors are formed of a memory material.

In some implementations, the memory material is nitinol.

In some implementations, a lasso-type device for preventing regurgitation at a native heart valve includes an encircling element capable of encircling native chordae tendineae, and a clip affixed to the encircling element capable of permanently securing the encircling element.

In some implementations, a constriction device for preventing regurgitation at a native heart valve includes a plurality of tissue anchors capable of being secured into native heart tissue, and a constriction element joining the plurality of tissue anchors.

In some implementations, the plurality of tissue anchors and the constriction element are constructed of a biocompatible material.

In some implementations, the biocompatible material is selected from stainless steel, nitinol, and titanium.

In some implementations, the tissue anchors comprise a corkscrew-like element connected to a screw head.

In some implementations, the lasso-type device further includes a ratcheting mechanism, a spool, or a winch for tightening the constriction element.

In some implementations, the constriction element is selected from a cable, a strut, or a suture.

The methods of treatment herein can be performed on a living animal or on a non-living cadaver, cadaver heart, simulator (e.g., with the body parts, tissue, etc. being simulated), anthropomorphic ghost, etc.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 illustrates a schematic cross-sectional view of a human heart.

FIG. 2 illustrates a schematic top view of a mitral valve annulus of a heart.

FIGS. 3A-3C illustrate examples of occluding or obstructing devices with a coil.

FIGS. 4A-6D illustrate example methods to install or deploy occluding or obstructing devices with a coil.

FIG. 7 illustrates examples of occluding or obstructing devices possessing hook-like anchors.

FIGS. 8A-8C illustrate example methods to install or deploy occluding or obstructing devices possessing hook-like anchors.

FIG. 9 illustrates an example lasso-type cinching device.

FIG. 10 illustrates an example inflow-side constriction device.

### DETAILED DESCRIPTION OF THE DISCLOSURE

Disclosed herein are various systems, apparatuses, methods, etc., including occluding or obstructing devices, which can be used to prevent valvular regurgitation at commissures or clefts of native heart valves. Additional implementations can be used in conjunction with expandable prosthetic valves (e.g., transcatheter heart valves (THV)) at a native valve annulus (e.g., mitral or tricuspid valve annulus), in order to prevent paravalvular leakage that may continue to exist after placement of a prosthetic valve and/or docking device for a prosthetic valve.

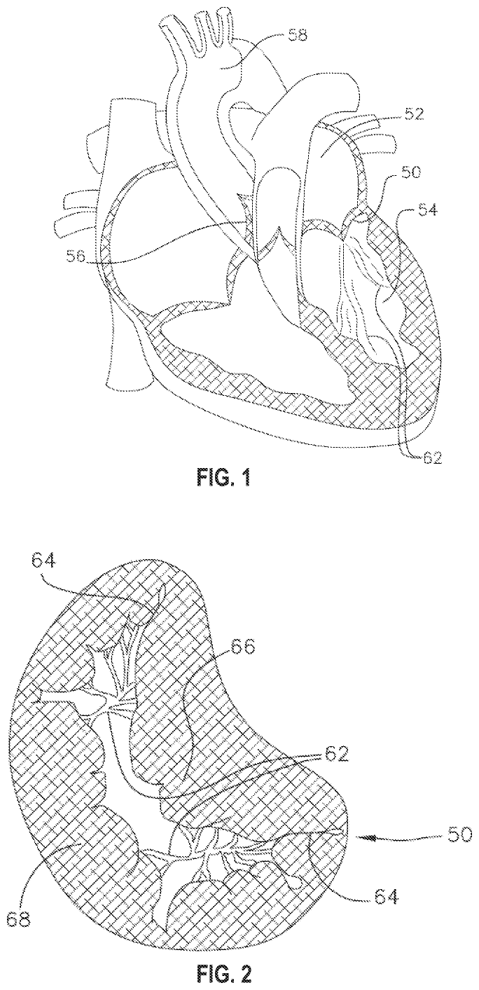

Referring first to FIGS. 1 and 2, the mitral valve 10 controls the flow of blood between the left atrium 12 and the left ventricle 14 of the human heart. After the left atrium 12 receives oxygenated blood from the lungs via the pulmonary veins, the mitral valve 10 permits the flow of the oxygenated blood from the left atrium 12 into the left ventricle 14. When the left ventricle 14 contracts, the oxygenated blood that was held in the left ventricle 14 is delivered through the aortic valve 16 and the aorta 18 to the rest of the body. Meanwhile, the mitral valve should close during ventricular contraction to prevent any blood from flowing back into the left atrium.

When the left ventricle contracts, the blood pressure in the left ventricle increases substantially, which serves to urge the mitral valve closed. Due to the large pressure differential between the left ventricle and the left atrium during this time, a large amount of pressure is placed on the mitral valve, leading to a possibility of prolapse, or eversion of the leaflets of the mitral valve back into the atrium. A series of chordae tendineae 22 therefore connect the leaflets of the mitral valve to papillary muscles located on the walls of the left ventricle, where both the chordae tendineae and the papillary muscles are tensioned during ventricular contraction to hold the leaflets in the closed position and to prevent them from extending back towards the left atrium. This helps prevent backflow of oxygenated blood back into the left atrium. The chordae tendineae 22 are schematically illustrated in both the heart cross-section of FIG. 1 and the top view of the mitral valve of FIG. 2.

A general shape of the mitral valve and its leaflets as viewed from the left atrium is shown in FIG. 2. Commissures 24 are located at the ends of the mitral valve 10 where the anterior leaflet 26 and the posterior leaflet 28 come together. Various complications of the mitral valve can potentially cause fatal heart failure. One form of valvular heart disease is mitral valve leak or mitral regurgitation, characterized by abnormal leaking of blood from the left ventricle through the mitral valve back into the left atrium. This can be caused, for example, by dilation of the left ventricle causing the native mitral leaflets to not coat completely, resulting in a leak, by damage to the native leaflets, or weakening of (or damage to) the chordae tendineae and/or papillary muscles. In these circumstances, it may be desirable to repair the mitral valve.

### Occluding Devices

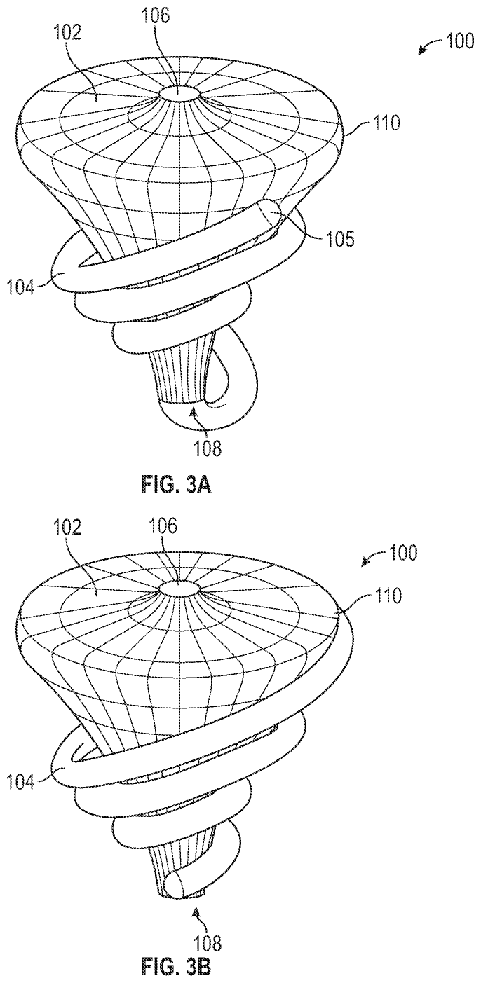

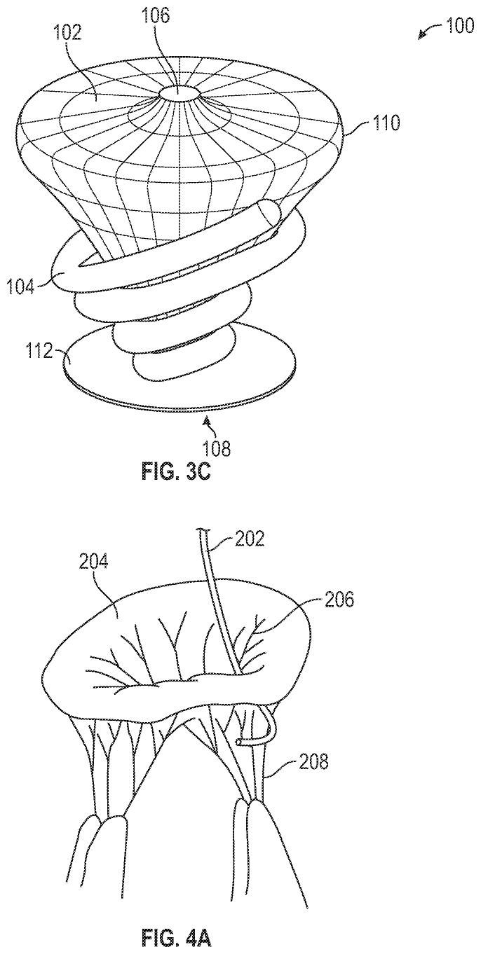

Turning to FIGS. 3A-3C, examples of an occluding or obstructing devices 100 for preventing valvular regurgitation and/or valvular prolapse are illustrated. FIGS. 3A-3C illustrate examples possessing a central plug 102 along with a coil 104 to surround native chordae tendineae (“chordae”). Coil 104 can include ends 105 that are rounded or domed. Rounded or domed ends may be beneficial to limit sharp or blunt edges that may cause damage to native tissues. Additionally, rounded or domed ends 105 may allow coil 104 to deflect around tissue, such as chordae, when being deployed.

In some implementations, coil 104 possesses at least one turn encircling central plug 102. In some implementations, the at least one turn extends around the central axis of central plug 102. In some implementations, the at least one turn is at least 2 turns (e.g., at least 720° of rotation), at least 3 turns (e.g., at least 1080° of rotation), or as many turns to provide sufficient retention force of the central plug against native anatomy (e.g., chordae tendineae and/or leaflets). In some implementations coil 104 is sized to encircle central plug 102 and at least some of the native chordae tendineae near the commissure of a native heart valve.

In some implementations, the central plug 102 possess a generally conical shape to arrest valvular prolapse. In some implementations, central plug 102 traverses a native valvular annulus. Further, these examples possess a proximal side 106, which refers to a portion of the occluding device 100 at the inflow side of a valve (e.g., atrial side for a mitral valve), and a distal side 108, which refers to a portion of the occluding device 100 at the outflow side of a valve (e.g., ventricular side for a mitral valve). In some implementations, proximal side 106 defines a larger or broader end, or “base,” of a generally conical shape, and distal side 108 defines a narrow end, or “tip,” opposite the base or proximal side 106. In some implementations, the central plug 102 defines a central axis running longitudinally through central plug 102 between the proximal side 106 (or base) and distal side 108 (or tip).

FIG. 3A illustrates an example, where the coil 104 and central plug 102 are singular unit connected at the distal side 108, while FIG. 3B illustrates a singular unit example where the coil 104 and central plug 102 are connected on the outer diameter 110 of the proximal side 106 of the central plug 102. The single piece examples in FIGS. 3A-3B prevent slipping, dislodgement, or displacement of the central plug 102 from a coil 104. On the other hand, FIG. 3C illustrates a two-part example where the coil 104 and central plug 102 are individual pieces. In such examples, a distal flange 112 can be utilized to prevent slipping, dislodgement, or displacement of the central plug 102 from coil 104. While FIG. 3C illustrates a flanged portion, variations on this general structure, including the use of a more symmetrical or hourglass shape of the central plug 102, where a constriction or waist prevents a coil 104 from slipping or migrating. While FIGS. 3A-3C illustrate approximately three encircling turns on coil 104, various implementations can possess any number of turns sufficient to maintain a retention force on the central plug 102 to prevent slipping, dislodgement, or displacement of an occluding device 100.

In some implementations, such as in FIGS. 3A-3C, the occluding devices 100 are made of a resilient material that is capable of being compacted on or in a catheter for delivery. In some implementations, the occluding devices 100 are made of and/or comprises a memory material that can be compressed or manipulated and return to a specific shape once a force is removed. An example of a memory material is nitinol (or NiTi), but other shape memory alloys or shape memory metals can be used. The memory material can be formed into a weave (or braided weave) or a frame that is compressible and returns to its formed shape (e.g., generally conical central plug 102 and/or coil 104) once released from a catheter. Furthermore, braiding or structuring from the weave on a coil 104 and central plug 102 can allow for additional retention forces due to the interactions in the textures.

Some implementations incorporate a biocompatible and/or atraumatic material as a covering on a central plug 102 to prevent damage to native tissue, including ePTFE, bovine pericardium, porcine pericardium, equine pericardium, woven PTFE, knitted PTFE, braided PTFE, polyurethane, electrospun ePTFE, dipped thermoplastic, sprayed thermoplastic, other organic tissues, other non-organic tissues, and combinations thereof. Furthermore, semipermeable or impermeable material can be disposed on a central plug 102 to prevent flow and/or encourage thrombosis such to prevent flow because of valvular regurgitation and/or prolapse. Some implementations utilize materials and/or compounds to encourage tissue ingrowth into a central plug 102.

In some implementations, occluding device 100 possesses a lubricous outer surface. In some implementations, the lubricous outer surface is a hydrophilic coating or a slick jacket to reduce friction between an occluding device 100 and native tissue (e.g., chordae). In some implementations, the lubricous outer surface is disposed on the coil 104, while some examples dispose the lubricous outer surface on the central plug 102. Some implementations dispose the lubricous outer surface on both the coil 104 and the central plug 102. In some implementations, this is accomplished with a temporary lubricous sleeve or sheath that can be placed over occluding device 100 during delivery, and which is retractable from off of occluding device 100 after occluding device 100 is in a desired position/location. In some implementations, the lubricous coating is constructed of a temporary and/or bioabsorbable material, such that after a period of time the coating will disappear, preventing slippage, repositioning, or other movement of an occluding device after deployment or installation. Such temporary materials can dissipate as a factor or function of time when exposed to body temperatures and/or fluids, while some examples allow for a dissolution via introduction of a biocompatible solvent or other agent to increase the dissolution rate of the lubricous coating. In some implementations, a lubricous or low-friction sleeve/sheath is incorporated into a transvascular and transcatheter delivery system.

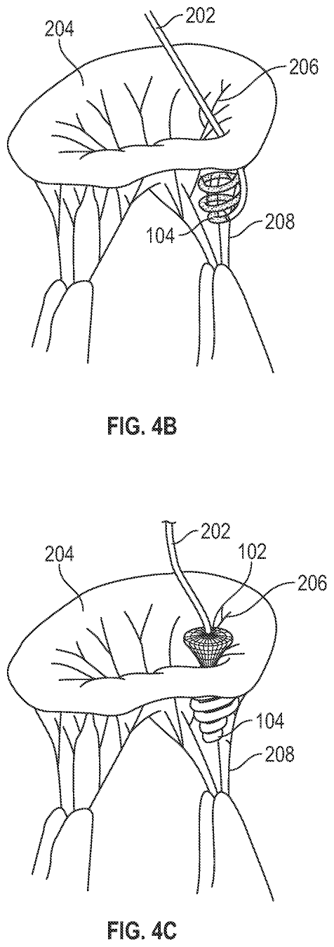

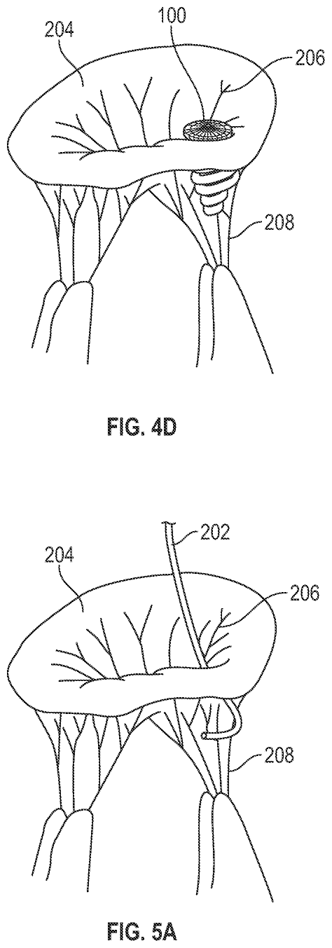

Turning to FIGS. 4A-4D, an example installation of an occluding device 100 is illustrated, where the occluding device is a single-piece occluding device anchored at the distal tip (e.g., the example of FIG. 3A). As seen in FIG. 4A, a delivery catheter 202 is navigated through an annulus 204 at or near a commissure 206. In FIG. 4B, once a delivery catheter 202 is navigated to the outflow side of a valve (e.g., ventricular side of mitral valve) a coil 104 is expelled from the catheter and allowed to encircle chordae 208. As a coil 104 is being expelled, delivery catheter 202 may be pushed further into the outflow side to allow proper encircling of the chordae 208. In FIG. 4C, upon delivery of the coil 104, a central plug 102 can be expelled from the delivery catheter 202 and allowed to expand within the coil 104 and within the chordae 208, such that the chordae 208 are held between the central plug 102 and coil 104. A fully installed occluding device 100 is illustrated at a commissure 206 in FIG. 4D, such that a proximal portion 106 of the occluding device 100 is placed at or above the annular plane to prevent or limit commissural regurgitation and/or prolapse.

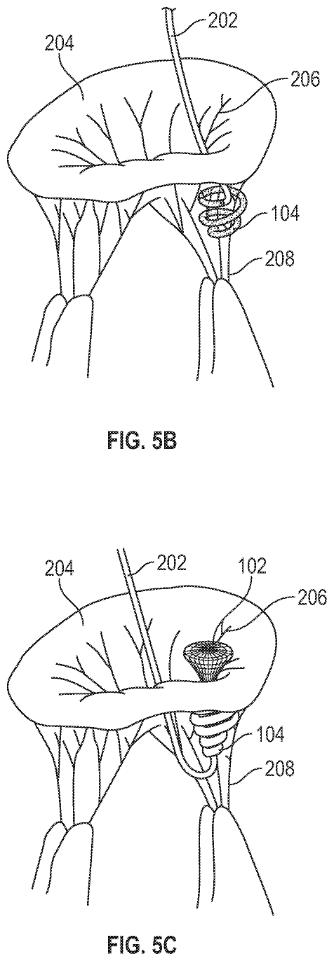

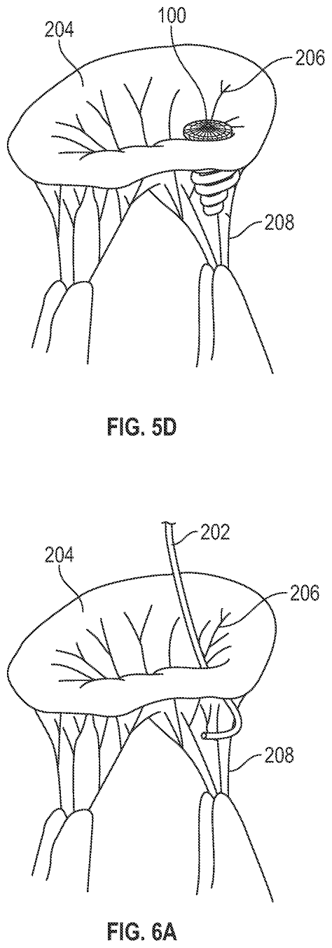

FIGS. 5A-5D illustrate a similar method of delivery of single-piece occluding device anchored at a proximal position (e.g., the example of FIG. 3B). As seen in FIG. 5A, a delivery catheter 202 is navigated through an annulus 204 at or near a commissure 206. In FIG. 5B, once a delivery catheter 202 is navigated to the outflow side of a valve (e.g., ventricular side of mitral valve) a coil 104 is expelled from the catheter and allowed to encircle chordae 208. As a coil 104 is being expelled, delivery catheter 202 can be retracted through the annulus 204 to allow proper encircling of the chordae 208. In FIG. 5C, upon delivery of the coil 104, a central plug 102 can be expelled from the delivery catheter 202 and allowed to expand within the coil 104 and within the chordae 208, such that the chordae 208 are held between the central plug 102 and coil 104. A fully installed occluding device 100 is illustrated at a commissure 206 in FIG. 5D, such that a proximal portion 106 of the occluding device 100 is placed at or above the annular plane to prevent or limit commissural regurgitation and/or prolapse.

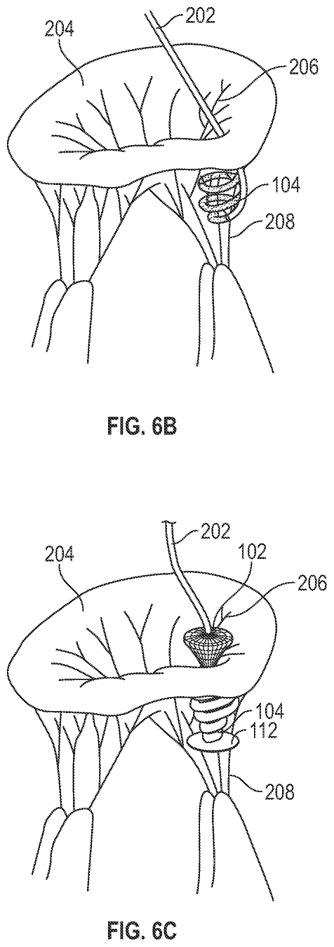

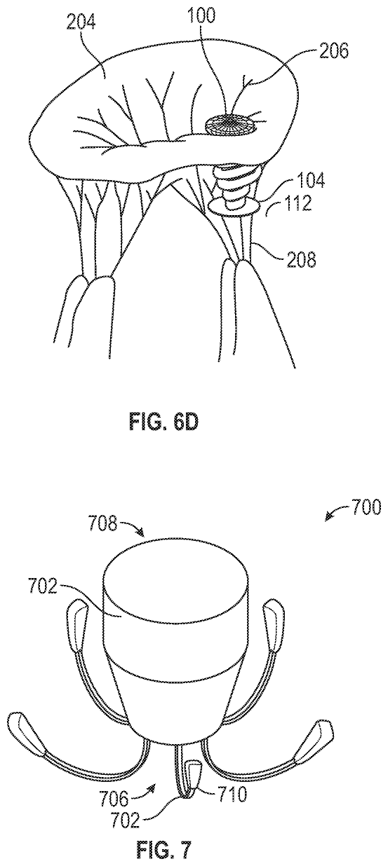

Additionally, FIGS. 6A-6D illustrate a similar method of delivery of two-piece occluding device (e.g., the example of FIG. 3C). As seen in FIG. 6A, a delivery catheter 202 is navigated through an annulus 204 at or near a commissure 206. In FIG. 6B, once a delivery catheter 202 is navigated to the outflow side of a valve (e.g., ventricular side of mitral valve) a coil 104 is expelled from the catheter and allowed to encircle chordae 208. As a coil 104 is being expelled, delivery catheter 202 can be retracted toward the annulus 204 or pushed further into the outflow side to allow proper encircling of the chordae 208. In FIG. 6C, upon delivery of the coil 104, a central plug 102 can be expelled from the delivery catheter 202, which is allowed to expand within the coil 104 and within the chordae 208, such that the chordae 208 are held between the central plug 102 and coil 104. In FIG. 6D, a fully installed occluding device 100 is illustrated at a commissure 206, such that a proximal portion 106 of the occluding device 100 is placed at or above the annular plane to prevent or limit commissural regurgitation and/or prolapse. While FIGS. 6A-6D illustrate a single delivery catheter 202, some examples utilize a second delivery catheter for the central plug 102, where a single delivery catheter may be difficult to construct or utilize for the delivery and installation of both a coil 104 and central plug 102.

Additionally, while FIGS. 4A-6D illustrate methods to install coil 104 prior to installation of a central plug 102, some examples can include installing the central plug 102 prior to installation of a coil 104, which may allow proper placement of the central plug 102 prior to securing it with a coil 104. Such implementations would be understood to one of skill in the art based on the illustrations shown in FIGS. 4A-6D, where the central plug would be installed within a delivery catheter 202 first and installed prior to the encircling of chordae 208 by a coil 104.

Turning to FIG. 7, an example of an occluding device 700 possessing distal anchors is illustrated. Occluding device 700 possesses a central plug 702 with a generally rounded shape to allow for occlusion or obstruction of a valve commissure. These examples also possess a plurality of hook-like anchors 704 to anchor occluding device 700 at a native annulus. In such examples, the hook-like anchors 704 extend from a distal or outflow side 706 of occluding device 700 to capture valvular leaflets against central plug 702. Hook-like anchors 704 extend radially from a central axis of an occluding device 700, wherein a central axis is defined as an axis formed between the distal side 706 and a proximal or inflow side 708 of an occluding device 700. In some implementations, hook-like anchors 704 further comprise a contoured shape to be atraumatic to native tissue. In some implementations, the contoured shape 710 is a covering disposed on anchors 704 comprised of a soft and/or formable material.

In some implementations an occluding device 700 is constructed of a memory metal, such as described above in relation to FIGS. 3A-3C. However, some examples can utilize a biocompatible and/or atraumatic materials, such as a foam or fabric material (such as described above) for the central plug 702, including permeable and semipermeable materials, to allow for occlusion of a native annulus. Some implementations can also utilize materials and compounds to encourage tissue ingrowth into central plug 702.

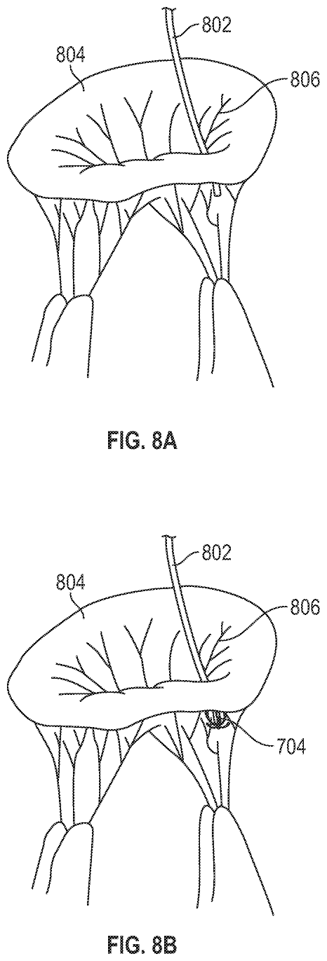

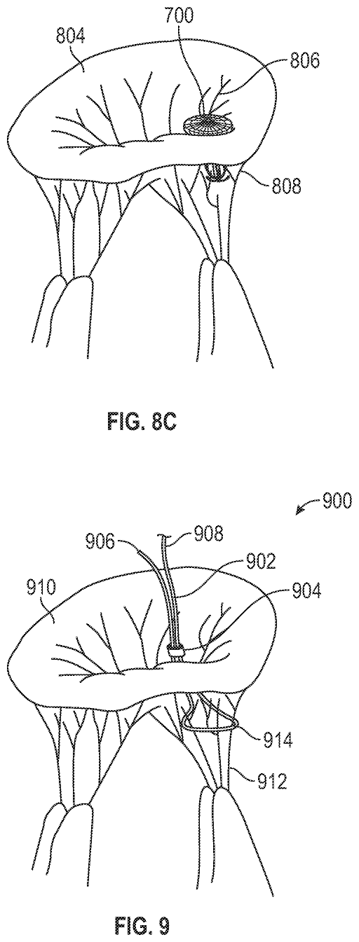

Turning to FIGS. 8A-8C, an example method to delivery an occluding device with hook-like anchors (e.g., the example of FIG. 7) is illustrated. In FIG. 8A, a delivery catheter 802 through an annulus 804 at or near a commissure 806. In FIG. 8B, once a delivery catheter 802 is navigated to the outflow side of a valve (e.g., ventricular side of mitral valve) hook-like anchors 704 are expelled from the delivery catheter 802. Delivery catheter can be retracted toward the annulus 804 to allow hook-like anchors 704 to anchor against native leaflets 808. Upon placement of hook-like anchors 704, central plug 702 can be expelled from delivery catheter 802, resulting in a fully deployed occluding device 700, as illustrated in FIG. 8C. In FIG. 8C, a proximal portion 708 of the occluding device 700 is placed at or above the annular plane to prevent or limit commissural regurgitation and/or prolapse.

### Lasso-Type Cinching Device

Turning to FIG. 9, an example lasso-type mechanism 900 is illustrated at a native heart valve. In some implementations, lasso-type mechanisms 900 possess an encircling element 902, such as string, suture, or other flexible and free material capable of encircling tissue (e.g., chordae tendineae) and a clip 904 affixed to encircling element 902. The encircling element 902 possesses a distal or free end 906 and a proximal or anchored end 908. In some implementations, the encircling element 902 is delivered to a native valve 910 via a delivery catheter (not shown), whereupon a distal end 906 encircles chordae 912 or other native tissue. Upon encircling chordae 912, distal end 906 is recaptured by the delivery catheter, and clip 904 is used to cinch, or tighten, a loop 914 created around chordae 912. Upon cinching the loop 914, clip 904 can be locked in place via crimping, an adhesive, or any other method for permanently fixing or securing a clip around encircling element 902. Some implementations can further sever extra lengths of distal 906 and proximal 908 ends of encircling element 902.

### Inflow-Side Constriction Device

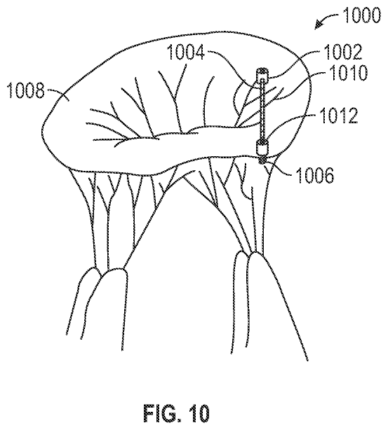

Turning to FIG. 10, an example of a constriction device 1000 to constrict a commissure via an inflow side of a valve is illustrated. In some implementations, a constriction device 1000 comprises a plurality of tissue anchors 1002 comprising a screws or other anchoring mechanism capable of being secured into native heart tissue, such as an annulus, leaflets, or wall (e.g., atrial wall). Tissue anchors 1002 can be joined via a constriction element 1004, such as a cable, strut, suture, or other element capable of joining tissue anchors 1002. Some implementations are constructed of a biocompatible material capable, such as stainless steel, nitinol, titanium, or any other suitable material.

To install some examples, tissue anchors 1002 can comprise corkscrew like elements 1006 which can be installed into an annulus 1008 near a commissure 1010. Alternatively, some embodiments install tissue anchors 1002 into an atrial or a ventricular wall. In corkscrew-like elements 1006, the anchors can be installed via a suitable tool that mates with a screw head 1012 (e.g., hex or star). In some embodiments, screws allow for removal or replacement of a constriction device 1000. However, some embodiments may be a permanent solution via barbs or some other mechanism that prevents the removal of a constriction device 1000. Upon installation of tissue anchors 1002, constriction element 1004 can be tightened via a ratcheting mechanism, spool, or winch that allows for tissue anchors 1002 to be drawn closer to each other, thus bringing valvular leaflets in closer proximity to each other. However, some implementations can have a static length on the constriction element 1004, such that installation of a second tissue anchor is the action that brings leaflets in closer proximity to each other, and no additional action is necessary to tighten or constrict constriction element 1004.

### Sterilization

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

Further, the methods of treatment herein can be performed on a living animal or on a non-living cadaver, cadaver heart, simulator (e.g., with the body parts, tissue, etc. being simulated), anthropomorphic ghost, etc.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In the context of the present application, the terms “lower” and “upper” are used interchangeably with the terms “inflow” and “outflow”, respectively. Thus, for example, the lower end of the valve is its inflow end and the upper end of the valve is its outflow end.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device toward the user, while distal motion of the device is motion of the device away from the user. The terms “longitudinal” and “axial” refer to an axis extending in the proximal and distal directions, unless otherwise expressly defined.

In view of the many possible embodiments to which the principles of the disclosed technology can be applied, it should be recognized that the illustrated embodiments are only preferred examples and should not be taken as limiting the scope of the disclosure. Rather, the scope of the disclosure is at least as broad as the following claims.

What is claimed is:

1. An occluding device for preventing regurgitation or prolapse at a native heart valve, comprising:
a central plug having a generally conical shape, wherein the generally conical shape defines a proximal side defining a base of the generally conical shape and a distal side defining a narrow end of the generally conical shape and opposite the base, and a central axis running between the distal and proximal sides; and
a coil possessing at least one turn extending along the central axis; wherein the coil is sized to encircle native chordae tendineae of a heart valve and provide a retention force against the central plug.

2. The occluding device of claim 1, wherein the coil is joined to the central plug at the distal side.

3. The occluding device of claim 1, wherein the coil is joined to the central plug at an outer diameter of the proximal side.

4. The occluding device of claim 1, wherein the central plug further comprises a flange extending from the distal tip.

5. The occluding device of claim 1, wherein the central plug traverses a native annulus, such that the proximal side resides on the inflow side of a native annulus, and the distal side resides on the outflow side of the native annulus.

6. The occluding device of claim 1, wherein the coil possesses at least two turns.

7. The occluding device of claim 1, wherein the central plug comprises a covering.

8. The occluding device of claim 7, wherein the covering comprises a biocompatible material or atraumatic material.

9. The occluding device of claim 7, wherein the covering is constructed of ePTFE, bovine pericardium, porcine pericardium, equine pericardium, woven PTFE, knitted PTFE, braided PTFE, polyurethane, electrospun ePTFE, dipped thermoplastic, sprayed thermoplastic, other organic tissues, other non-organic tissues, and combinations thereof.

10. The occluding device of claim 7, wherein the covering comprises a material or compound to encourage tissue ingrowth.

11. The occluding device of claim 1, wherein the coil and the central plug possess a braided weave.

12. The occluding device of claim 1, further comprising a lubricous outer surface.

13. The occluding device of claim 12, wherein the lubricous outer surface is disposed on the coil.

14. The occluding device of claim 12, wherein the lubricous outer surface is disposed on the central plug.

15. The occluding device of claim 12, wherein the lubricous outer surface is constructed of a bioabsorbable material.

16. An occluding device for preventing regurgitation at a native heart valve, comprising:
a central plug having a generally conical shape, wherein the generally conical shape defines a proximal side being the base of the conical shape and a distal side defining the narrow end opposite the base and a central axis running between the distal and proximal sides; and
a plurality of tissue anchors extending radially from the distal side and sized to capture valvular leaflets against the central plug.

17. The occluding device of claim 16, wherein the plurality of tissue anchors possess a contoured shape.

18. The occluding device of claim 17, wherein the contoured shape comprises a covering disposed upon the plurality of tissue anchors.

19. The occluding device of claim 16, wherein the central plug comprises a biocompatible material or atraumatic material.

20. The occluding device of claim 19, wherein the biocompatible or atraumatic material or covering is selected from: ePTFE, bovine pericardium, porcine pericardium, equine pericardium, woven PTFE, knitted PTFE, braided PTFE, polyurethane, electrospun ePTFE, dipped thermoplastic, sprayed thermoplastic, other organic tissues, other non-organic tissues, and combinations thereof.
